(19) United States
(12) Patent Application Publication
(10) Pub. No.: US 2010/0331963 A1
(43) Pub. Date: Dec. 30, 2010

(54) Method of Making Suture-Less Hollow Scaffolds

(76) Inventors: Jackie Donners, West Windsor, NJ (US); Dhanuraj S. Shetty, Jersey City, NJ (US); Mark D. Timmer, Jersey City, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/495,001

(22) Filed: Jun. 30, 2009

(51) Int. Cl. A61F 2/06; B29C 71/02; A61F 2/04

(52) U.S. Cl. 623/1.38; 264/237; 623/23.71

(57) ABSTRACT

A method of making a hollow organ tissue engineering scaffold for repairing organs. Specifically, the hollow organ tissue engineering scaffold is made from a nonwoven fabric having first and second biocompatible materials, wherein the first material has a lower melting temperature than the first, and the first material is at least partially melted to form the scaffolds.

### CROSS-REFERENCE TO RELATED APPLICATIONS

This application is related to commonly assigned patent application Ser. No. ______ (Applicants' Docket No. RTX-5014) filed on evendate herewith, which is incorporated by reference

### FIELD OF THE INVENTION

This invention is related to methods of making tissue engineering scaffolds, more specifically, methods of making tissue engineering scaffolds for the repair and regeneration of hollow anatomic structures.

### BACKGROUND OF THE INVENTION

Human organs are exposed to a variety of possible injuries starting from the time of fetus development. Individuals may suffer from congenital disorders, cancer, trauma, infection, inflammation or other conditions that may lead to organ damage, or organ loss, and require eventual reconstruction and replacement. Organ damage may result in some type or degree of loss of function. Whenever there is a deficient organ function, replacement, partial replacement, or augmentation may be achieved with artificial organs or organ transplantation. There are obvious disadvantages to this process since an artificial organ is not able to replicate all the functionalities of the organ it is supposed to replace, and because of organ donor shortages and immunologic rejections. Tissue-engineering therapies have been utilized to achieve the goal of reproducing and replacing such functionalities. A key concept in tissue engineering (TE) is the use of material-based porous scaffolds to provide physical support and a local environment for cells to enable and facilitate tissue development. The scaffolds serve complex functions of guiding cellular behavior in different TE applications. Scaffolds can be seeded with embryonic adult stem cells, progenitor cells, mature differentiated cells, co-cultures of cells or minced tissue derived from autologous, allogenic & xenogenic source to induce tissue formation in vitro and in vivo. Scaffolds serve as temporary substrates for supporting and guiding tissue. Such tissue engineering techniques ideally result in the regeneration of the desired tissue, while the scaffold is eventually completely resorbed by the body. It is known to use tissue engineering techniques to regenerate various tissues and organs such as, skin, bladder, cartilage, meniscus and the like.

Since the mid-1980's researchers have developed many novel techniques to shape polymer-based scaffolds into complex architectures that exhibit the desired properties for specific tissue-engineering applications. Techniques have been utilized to transform a two-dimensional substrate and convert it into a three dimensional scaffold. One of the know techniques employed for bladder reconstruction, utilizes a flower shaped petal prepared from nonwoven fabric, that is absorbable and woven into a three dimensional shape (See FIG. 1). The petal-shaped nonwoven material is then sutured to form a three-dimensional construct. In order to impart appropriate stiffness to the woven or sutured structures are dip coated in a 5% (w/v) 50/50 poly(lactide-co-glycolide) (PLA/PGA) (M_{w}=40,000-70,000) solution in dichloromethane (DCM). There are several drawbacks and deficiencies associated with this known process, including that the suturing step is labor intensive and highly operator dependent. Furthermore, the coating process creates a gradient of a coating layer and uses organic solvent which is undesirable for manufacturing purposes. This manufacturing process is also time consuming.

Accordingly, there exists a need in this art for methods for novel methods of fabricating three-dimensional hollow anatomic scaffolds for use in tissue engineering procedures that overcome these deficiencies.

### SUMMARY OF THE INVENTION

We have disclosed herein a method of making a hollow organ tissue engineering scaffold. The method includes the steps of providing a nonwoven fabric comprising a first biocompatible, bioabsorbable material having a first melting temperature and a second biocompatible, bioabsorbable material having a second melting temperature, wherein the first melting temperature is lower than the second melting temperature, forming the nonwoven fabric into a hollow organ shape, heating the three dimensional shaped fabric to a temperature sufficient to at least partially melt the first biocompatible, bioabsorbable material and without melting the second biocompatible, bioabsorable material, and allowing the three dimensional shaped fabric to cool to room temperature, thereby providing a hollow organ tissue engineering scaffold.

Yet another embodiment of the present invention is a hollow organ tissue engineering scaffold made by the afore-described process.

These and other aspects and advantages of the present invention will become more apparent from the following description and accompanying drawings.

### BRIEF DESCRIPTION OF THE DRAWINGS

FIG. 1 is a schematic flow diagram of a process of the prior art for making bladder augmentation tissue engineering scaffolds.

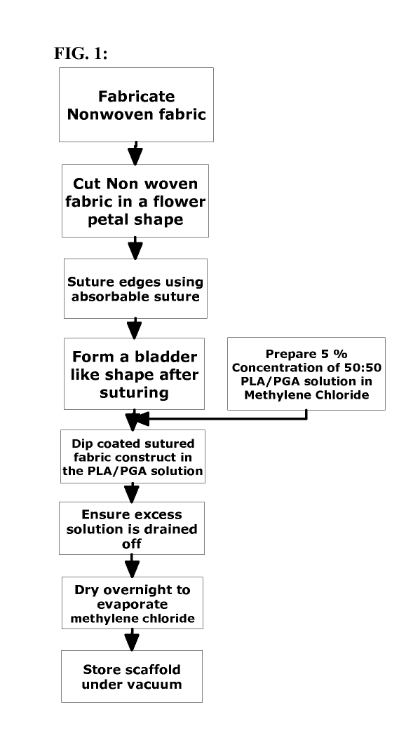

FIG. 2 is a schematic flow diagram of one embodiment of the process of the present invention for preparing hollow organ tissue engineering scaffolds

FIG. 3 is a schematic flow diagram of an alternative embodiment of the process of the present invention for preparing hollow organ tissue engineering scaffolds

FIG. 4 is a scanning electron micrograph of 70:30 (90/10 poly(glycolide-co-lactide/poly(p-dioxanone) nonwoven melt bonded with poly(p-dioxanone) film with no load applied.

FIG. 5 shows scanning electron micrographs of an exemplary hollow organ tissue engineering scaffold prepared from 90/10 poly(glycolide-co-lactide/poly(p-dioxanone) nonwovens.

FIG. 6 is a graph that shows DNA content after cell culture on a dip coated (prior art method) scaffold, melt-bonded scaffold, and unprocessed nonwoven fabric

### DETAILED DESCRIPTION OF THE INVENTION

For the purpose of this invention, the term “hollow organ tissue engineering scaffold” is defined as a scaffold having suitable shape for repair or regeneration of hollow organ structures. The hollow organ scaffold may have any suitable shape such that there is at least one opening for attachment to the hollow organ for repair or regeneration. Hollow organ structures include but are not limited to bladder, urethra, jejunum, esophagus, trachea, colon, blood vessels, stomach, and nerve guides. The hollow organ may be at least partially available for surgical attachment. Alternatively, in some cases, the hollow organ can be completely absent.

As used herein, the term “nonwoven fabric” includes, but is not limited to, bonded fabrics, formed fabrics, or engineered fabrics, that are manufactured by processes other than weaving or knitting. More specifically, the term “nonwoven fabric” refers to a porous, textile-like material, usually in flat sheet form, composed primarily or entirely of staple fibers assembled in a web, sheet or batt. For the purposes of this invention, staple fibers are cut to a specific length from the continuous filament fiber. Usually the staple fiber is cut to length in the range of about 1.5 inches to about 8 inches. The structure of the nonwoven_ fabric is based on the arrangement of, for example, staple fibers that are typically arrayed more or less randomly. The tensile, stress-strain and tactile properties of the nonwoven fabric ordinarily stem from fiber to fiber friction created by entanglement and reinforcement of, for example, staple fibers, and/or from adhesive, chemical or physical bonding. Notwithstanding, the raw materials used to manufacture the nonwoven fabric may be yarns, scrims, netting, braids or filaments made by processes that include, weaving or knitting.

Preferably, the nonwoven fabric is made by processes other than, weaving or knitting. For example, the nonwoven fabric may be prepared from yarn, scrims, netting or filaments that have been made by processes that include, weaving or knitting. The yarn, scrims, netting and/or filaments are crimped to enhance entanglement with each other and attachment to the second absorbable woven or knitted fabric. Such crimped yarn, scrims, netting and/or filaments may then be cut into staple fibers that is long enough to entangle. The staple fiber may be between about 0.1 and 3.0 inches long, preferably between about 0.75 and 2.5 inches, and most preferably between about 1.5 and 2.0 inches. In one embodiment, the staple fiber length is about 2 inches. The staple fibers may be carded, wet laid, or air laid to create a nonwoven batt, which may be then calendared, needlepunched, hydroentangled, or air entangled into the nonwoven_ fabric. Additionally, the staple may be kinked or piled. Other methods known in the art for the production of nonwoven fabrics may be utilized.

In one embodiment, the nonwoven fabric has a thickness in the range of about 0.5 mm to about 5 mm. In another embodiment, the nowoven fabric has a thickness in the range of about 0.5 mm-to about 2 mm. In one embodiment, the nonwoven fabric has a density in the range of about 60 mg/cc-about 300 mg/cc. In another embodiment the nonwoven fabric has a density in the range of about 60-120 mg/cc.

In one embodiment, the nonwoven fabric is comprised of a first biocompatible, bioabsorbable material having a first melting temperature and a second biocompatible, bioabsorbable material having a second melting temperature, wherein the first melting temperature is lower than the second melting temperature. The first and second melting temperatures must be sufficiently different such that upon heating to the first melting temperature the first biocompatible, bioabsorbable material is at least partially melted and the second biocompatible, bioabsorable material is not melted. By partially melted, we mean that the first material will flow and attach to the second material such that upon cooling the two materials will be bonded together.

Suitable biocompatible, bioabsorbable materials include, but not limited to aliphatic polyester polymers, copolymers, or blends thereof. In one embodiment the biocompatible, bioabsorbable material are aliphatic polyester polymers which are typically synthesized in a ring opening polymerization of monomers including, but not limited to, lactide (including L-, D-, meso and D, L mixtures and lactic acid), glycolide (including glycolic acid), epsilon-caprolactone, p-dioxanone (1,4-dioxan-2-one), and trimethylene carbonate (1,3-dioxan-2-one). In one embodiment, the first biocompatible, bioabsorbable material is poly(p-dioxanone) (PDS). In one embodiment the second biocompatible, bioabsorbable material material is selected from the group consisting of poly(glycolide) (PGA) and poly(glycolide-co-lactide) (PGA/PLA). In another embodiment, the second biocompatible, bioabsorbable material is a poly(glycolide-co-lactide) having a monomer mole ratio of 90/10 glycolide/lactide (90/10 PGA/PLA).

The first biocompatible, bioabsorbable material is present in the nonwoven in the amount of about 20% to about 90% by weight. In one embodiment, the first biocompatible, bioabsorbable material is present in the nonwoven in the amount of about 30% to about 50% by weight. Most preferably the first biocompatible, bioabsorbable material present in the nonwoven is in the amount of about 30% by weight.

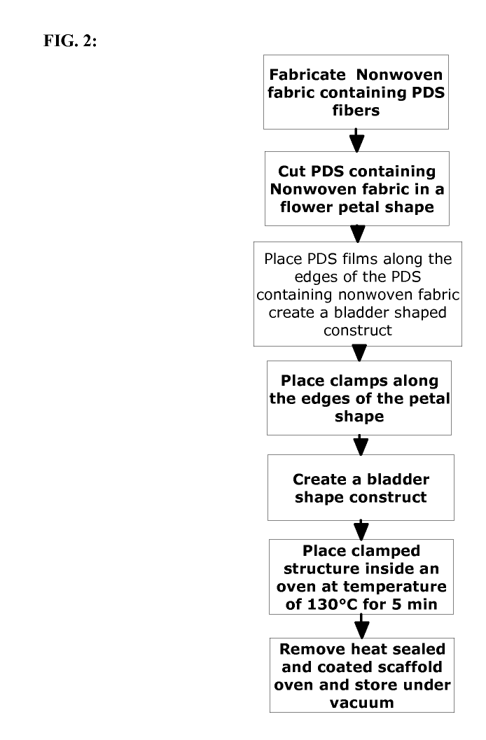

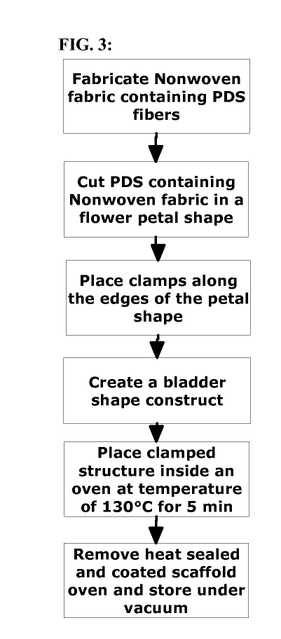

In the practice of the present invention, the nonwoven may be formed into the hollow organ shape by conventional methods such as, cutting the nonwoven into a suitable design and then approximating the edges of the nonwoven to form the hollow organ shape, placing the fabric in a suitable mold, and the like. Suitable designs to cut the nonwoven fabric into include but are not limited to square, rectangular, triangular, and petal. For example, for a bladder tissue engineering device the nonwoven may be cut into a flower petal design, approximate the adjacent edges, and temporarily hold the edges together to form a hollow, sphere shaped device with an opening at one end. The adjacent edges of the nonwoven may be held together by tacks, pins, clips, mechanical clamps that are designed to the contours of the overall scaffold edges, or any other device that secures the adjacent edges of the device until the next processing step is completed (see FIG. 2). Optionally, the adjacent edges may be further reinforced by placing a film comprising the first biocompatible, biodegradable polymer between the adjacent edges such that during the next step, the heating step, the edges are attached even more securely without affecting the porosity that is necessary for cell in growth in the rest of the scaffold (see FIG. 3). The hollow organ scaffold is now in a suitable shape for hollow organ repair including, but not limited to spherical, prism, and cylindrical.

The hollow organ shaped scaffold is then heated to at least partially melt the first biocompatible, bioabsorbable material. The scaffold may be heated by conventional means such as, a temperature controlled heated oven, vacuum oven, compression molding instrument or similar heating device. The scaffold is heated to a temperature for a time that is sufficient to effectively provide at least partial melting of the first biocompatible, bioabsorbable material such that the fibers become attached to each other upon cooling and provide structural integrity to the scaffold and without melting the second biocompatible, bioabsorbable material. Where PDS is the first biocompatible, bioabsorbable material the heating may be accomplished at a temperature in the range of about 105° C. to about 150° C. In one embodiment the heating may be accomplished at a temperature in the range of about 120° C. to about 140° C. In yet another embodiment, the heating may be accomplished at a temperature of about 130° C. The amount of time that the scaffold is heated will depend upon the applied temperature. The lower the temperature the more time that will be needed to melt the polymer and the higher the temperature the less time that will be needed to melt the polymer. Furthermore, if the heating is accomplished at lower temperatures, such as about 105° C. to about 120° C., for longer times the scaffold may need to be constrained in a mold to minimize contraction of the device. One of skill in the art would be able to select an appropriate time to heat the scaffold in order to partially melt the PDS fibers (or the first biocompatible bioabsorbable material). The hollow organ shaped scaffold is then allowed to cool to room temperature thereby providing a hollow organ tissue engineering scaffold.

Scaffolds can be implanted a cellular or with pre-applied cell populations, including for example minced tissue or other tissue. The cell or tissue sources can be autologous, xenogenic, or allogenic. In one embodiment, the source of the cells or minced tissue is autologous. Sources for the minced tissue include, but are not limited to bladder, urethra, blood vessels, lung, skin and the like. Cell populations that would be suitable for combination with the hollow organ tissue engineering scaffold include, but are not limited to endothelial cells, smooth muscle cells, stem cells, and the like.

The scaffolds of the present invention may be useful in treating organs. In particular, hollow organs, such as bladder, urethra, jejunum, esophagus, trachea, colon, blood vessels, stomach and other organs that may benefit from a hollow tissue engineering device such as nerve guides may benefit from placement of the present composite as a “patch” in an area requiring tissue augmentation or regeneration. For example, regarding the bladder, if an area of the bladder, is missing due to congenital defect or has been lost due to disease, injury or surgery (e.g., partial cystectomy), the patient may benefit from having the bladder area increased or restored to the original size as the particulars of the case allows.

In one embodiment, a hollow organ scaffold having a substantially spherical shape with an opening on one end may be useful for bladder repair after a partial cystectomy procedure. The partial cystectomy procedure is performed according to established methods. The sterile hollow organ scaffold is wetted using sterilie saline solution. The wet scaffold is then placed over the cystectomied bladder such that the opening of the scaffold is sutured over it using appropriate absorbable suture material. The scaffold and bladder is then covered with and attached to the omentum using fibrin glue. The surgical procedure is completed by closing the surgical site.

In the case of an cellular approach, where for example minced tissue is used, the minced tissue is disposed on the outer surface of the scaffold, the inner surface of the scaffold, or both prior to attaching the scaffold to the cystectomied bladder. The minced tissue can be obtained using any of a variety of conventional techniques, such as for example, by biopsy or surgical removal. Preferably, the tissue sample is obtained under aseptic conditions. Once a sample of living tissue has been obtained, the sample can then be processed under sterile conditions to create a suspension having at least one minced, or finely divided, tissue particle. The particle size and shape of each tissue fragment can vary, for example, the tissue size can be in the range of about 0.1 and 3 mm³, in the range of about 0.5 and 1 mm³, in the range of about 1 to 2 mm³, or in the range of about 2 to 3 mm³, but preferably the tissue particle is less than 1 mm³. The shape of the tissue fragments can include slivers, strips, flakes or cubes as examples. Some methods include mechanical fragmentation or optical/laser dissections. As mentioned previously, other sources of cell populations may be used in the practice of the present invention including stem cells and allographic tissue.

The following examples are illustrative of the principles and practice of the present invention, although not limited thereto.

### EXAMPLE 1

Samples of nonwoven fabrics were manufactured in a conventional manner Lot numbers and specifications for these nonwovens are tabulated in Table 1.

TABLE 1
Lot numbers and specifications of nonwovens.

| | | Ratio | | |
|---|---|---|---|---|
| | Material | (wt | Density | Thickness |
| Lot number | Composition | %) | (mg/cc) | (mm) |
| MD00134-1 | 90/10 | 100 | 100 | 1 |
| | PGA/PLA | | | |
| MD00235-1 | 90/10 | 100 | 100 | 1 |
| | PGA/PLA | | | |
| MD00236-1 | 90/10 | 100 | 100 | 1 |
| | PGA/PLA | | | |
| MD00236-2 | 90/10 | 100 | 100 | 1 |
| | PGA/PLA | | | |
| MD00323-1 | (90/10 | 50/50 | 100 | 1 |
| | PGA/PLA)/PDS | | | |
| MD00324-1 | (90/10 | 60/40 | 100 | 1 |
| | PGA/PLA)/PDS | | | |
| MD00325-1 | (90/10 | 70/30 | 100 | 1 |
| | PGA/PLA)/PDS | | | |
| MD00348-1 | (90/10 | 70/30 | 100 | 1.5 |
| | PGA/PLA)/PDS | | | |
| MD00349-1 | (90/10 | 70/30 | 100 | 2 |
| | PGA/PLA)/PDS | | | |
| 5248-05-2 | (90/10 | 50/50 | 200 | 1 |
| | PGA/PLA)/PDS | | | |
| 5248-05-3 | (90/10 | 60/40 | 200 | 1 |
| | PGA/PLA)/PDS | | | |

The nonwoven fabric samples were scoured upon receipt by incubating in alcohol followed by ultrapure water. The samples were subsequently dried by blotting with sterile gamma wipes, drying for 10 minutes with cold air and overnight drying under vacuum. Samples were cut into coupons with scissors or punched with a cutting die #31268 (DV Die, Danvers, Mass.) using a Carver 2696 laboratory press (Carver, Wabash, Ind.) at 2 tons of pressure.

Prior art control samples were prepared by suturing a petal-shaped sample into bladder-shaped scaffolds with 90/10 PGA/PLA suture sold under the tradename VICRYL (4-0 suture, Ethicon, Inc., Somerville, N.J., J415H, Lot #ZH6093) with an average stitch density of 17 stitches/inch and a knot at every 5^{th} stitch. The prior art scaffolds were dip-coated 3 times in a 5 weight % (50/50) PLA/PGA solution (Sigma, St. Louis, Mo., P2191) in dichloromethane and air-dried in between coating steps. Scaffolds were sterilized with ethylene oxide.

### EXAMPLE 2

### Melt-Bonding With PDS Film

In order to selectively reinforce the edges of the scaffold construct, strips of PDS films were inserted between the edges of the scaffold. The PDS films were about 800 microns in thickness. The PDS films were extruded using a typical film extrusion process. The PDS pellets were fed into the hopper of the extruder. The temperature of the machine was set according to the melting temperature of PDS. The temperatures in the different zones of the extruder were set to 150° C. The polymer melt came out the die that was set to the desired thickness of 800 microns. The film was then air cooled to room temperature. The cooled PDS film was then cut to desired shape and size. Coupons with dimensions of 8×1 inches were cut from nonwoven MD00236-1 (Example 1). Coupons were cut in half lengthwise. Next, a piece of 0.8 mm thick PDS film was either positioned on top of adjacent nonwoven samples or sandwiched between overlapping nonwoven samples. The nonwoven samples were bonded together by placing on the preheated platens of a Carver 2696 laboratory press (Carver, Wabash, Ind.), melting the PDS at 130° C. for 5 minutes followed by allowing the samples to cool to room temperature. A minimal amount of pressure was applied with the press but this led to a three-fold reduction of thickness of the nonwoven. Therefore, another sample having PDS film sandwiched between overlapping nonwovens was heated with a 1 mm thick aluminum plate on top as a weight without applied load from the press. The weight of the plate was sufficient to create a bond but did not result in a reduction of thickness. The breaking strengths as tabulated in Table 2 show that sandwiching the film between nonwovens results in much stronger bonds compared to positioning the film on top of the adjacent edges of nonwoven scaffold.

TABLE 2
Breaking strengths of nonwoven bonded together with PDS films.

| Sample | Force (N) |
|---|---|
| (90/10 PGA/PLA), PDS film on top, load | 4.87 |
| (90/10 PGA/PLA), PDS film sandwiched, load | 131.45 |
| (90/10 PGA/PLA), PDS film sandwiched, no load | 189.29 |

### EXAMPLE 3

### Hollow Scaffolds Generated With PDS Film

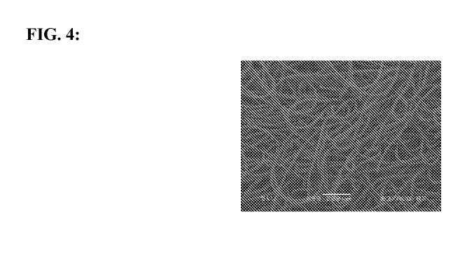

Two samples of MD00236-02 were punched with the cutting die as described in Example 1. For the first sample, pair of adjacent edges of the petal-shape were clamped together with two 2×1 cm paper binder clips with a small strip of 0.8 mm thick PDS film inserted in between adjoining edges to form the hollow shape. For the second sample, a petal was punched out of PDS film with the cutting die and overlaid with the nonwoven petal. Each pair of adjacent edges of the petal-shape were clamped together with 2 2×1 cm paper binder clips with the nonwoven side facing outward to form a hollow shape having the PDS film lining the inside. The constructs were placed over a mold and placed for 5 minutes in an oven pre-heated to 130° C. Samples were allowed to cool for 5 minutes, after which the binder clips were removed. Samples were stored under nitrogen until further use. Scanning electron microscopy (SEM) images were taken of the nonwoven side (outside) of the second sample. The image (FIG. 4) shows that the PDS film did not melt through and seal the pores of the nonwoven.

### EXAMPLE 4

### Melt-Bonding of (90/10 PGA/PLA)/PDS Nonwovens

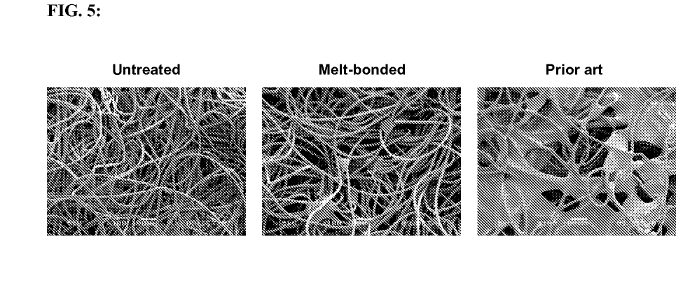

Experiments focused on finding optimal heating times and temperatures for melt-bonding of (90/10 PGA/PLA)/PDS nonwovens were performed on sample 5248-05-3 (Example 1). Coupons with dimensions of 8×1 inches were cut from the nonwoven. The coupons were then cut in half lengthwise. The samples were overlapped by 0.5-1 cm. The nonwoven samples were bonded together by placing on the preheated platens of a Carver 2696 laboratory press (Carver, Wabash, Ind.) with a 1 mm thick aluminum plate as a weight. An exposure time of five minutes at elevated temperature and five minutes of cooling at room temperature appeared to be sufficient. The initial experiments were performed at a press temperature of 130° C., well above the melting temperature of PDS (110° C.). An attempt was made to bond at 120° C., but samples did not form proper bonds and the edges of the material curled up. Apparently, at a 120° C. press temperature for 5 minutes the material was not fully melted and some contraction occurred. The scaffold required longer times at lower temperatures and needed to be constrained to prevent contraction. Heating the scaffold at 130° C. for 5 minutes was sufficient. Scanning electron microscopy (SEM) images were taken of the nonwoven surface The SEM image (FIG. 5) showed that the melt bonding resulted in a minimum amount of webbing, however the porosity of the sample was substantially unchanged compared to an untreated nonwoven, whereas the prior art process leads to a substantial amount of webbing therefore a reduction of porosity.

### EXAMPLE 5

### Effect of (90/10 PGA/PLA)/PDS Ratio on Stiffness

Next, the effect of (90/10 PGA/PLA)/PDS ratio on the material stiffness and breaking strength was evaluated. Ratios of 70/30, 60/40 and 50/50 by weight percent of 90/10 PGA/PLA fibers to PDS fibers were evaluated having a density of 100 mg/cc and a thickness of 1 mm. Coupons with dimensions of 6×1 inches were cut from samples MD00323-01, MD00324-01 and MD00325-1 (Example 1). The coupon was cut in half lengthwise and placed on a 1 mm thick aluminum plate with 1 cm overlap. A second aluminum plate was placed on top and the setup was put inside the Carver press pre-heated to 130° C. After 5 minutes, the water cooling was switched on and the samples were removed and stored under nitrogen. Breaking strengths were evaluated by clamping the two opposing edges into the grips of a mechanical testing instrument sold under the tradename INSTRON (Instron, Norwood, Mass.) and pulling them apart at a rate of 300 mm/min. As can be seen in Table 3 an increasing amount of PDS resulted in a larger thickness and larger breaking force. Also, the overall stiffness was greater, a result that is reflected in the fact that in the 50/50 case the bond (seal) broke rather than the nonwoven itself. The lower overall values are attributed to the fact that in these samples much less PDS was present. Wetting of the material did not lead to any appreciable loss of qualitative stiffness. For further studies, a 70/30 by weight of 90/10 PGA/PLA fibers to PDS fibers was chosen since a minimal amount of PDS is desirable from a biological perspective, e.g. the time it takes to completely degrade the scaffold.

TABLE 3
Thickness and breaking strengths of (90/10 PGA/PLA)/PDS
melt-bonded nonwovens with different (90/10 PGA/PLA)//PDS ratios.

| Ratio | Thickness (mm) | Break force (N) | Location of break |
|---|---|---|---|
| 70/30 | 0.50 | 40.82 | NW breaks |
| 60/40 | 0.56 | 47.36 | NW breaks |
| 50/50 | 0.67 | 55.28 | seal breaks |

### EXAMPLE 6

### Hollow Scaffolds Formed by Melt-Bonding of PDS/(90/10 PGA/PLA) Nonwovens

Samples of MD00323-01, MD00324-01, MD00325-1 and 5248-05-3 were punched with the cutting die (Example 1). Each edge of the petal-shape was clamped together with two 2×1 cm paper binder clips to form a hollow shape. The construct was placed over a mold and placed for 5 minutes in an oven pre-heated to 130° C. Samples were allowed to cool for 5 minutes, after which the binder clips were removed. Samples were stored under nitrogen until further use.

### EXAMPLE 7

### Mechanical Testing of Hollow Scaffolds

Breaking strengths of hollow scaffolds prepared in Examples 3 and 7 were evaluated by clamping the two opposing edges into the grips of an INSTRON and pulling them apart at 300 mm/min. The results are compared to two control samples: 1) prior art control sample (as prepared in Example 1); 2) nonwoven fabric (unprocessed) As can be seen in Table 5, sufficiently strong bonds, e.g. stronger or equal to all three control samples, were obtained in all cases.

TABLE 5
Breaking strength of hollow melt-bonded scaffolds

| | | Break | |
|---|---|---|---|
| Example | Sample | force (N) | Location of break |
| 3 | PDS film edge melt-bond | 183.85 | Seal/nonwoven |
| | PDS film petal melt-bond | 166.39 | Nonwoven |
| 7 | 50/50 (90/10 PGA/PLA)/PDS | 89.89 | Seal |
| | 60/40 (90/10 PGA/PLA)/PDS | 75.62 | Seal |
| | 70/30 (90/10 PGA/PLA)/PDS | 92.71 | Seal |
| Controls | Prior art control | 85.21 | Seal |
| | Non-treated nonwoven | 80.41 | |

### EXAMPLE 8

### In Vitro Cell Attachment/Viability Study

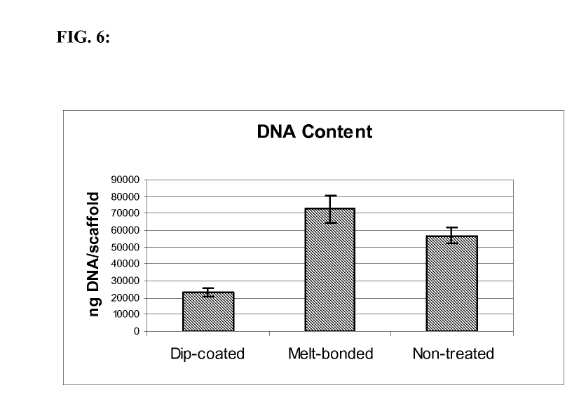

An in vitro study was performed to analyze differences in cell attachment and survivability of human fibroblasts on prior art scaffold (see Example 1) and melt bonded (90/10 PGA/PLA)/PDS nonwovens (70/30) as prepared in Example 6 compared to untreated 90/10 PGA/PLA nonwoven fabric (100 mg/cc, 1 mm thick). Four circular samples having a 6 mm diameter were punched from each of the nonwovens and one million human fibroblasts were seeded onto each sample and incubated at 37° C. One of each scaffold was assayed for cell viability after 24 hours with the LIVE/DEAD® assay (Invitrogen, Carlsbad, Calif.). Viabilities >95% were observed in all samples. The remaining 3 samples for each scaffold were cultured for 7 days after which the cells were lysed and their DNA content measured using the CyQuant Kit (Invitrogen, Carlsbad, Calif.). The results are graphed in FIG. 6. The DNA content analysis enables the determination of cell attachment and viability over a period of time following cell culturing. The DNA content for the prior art nonwoven samples was about 40% of the untreated sample. The melt bonded sample had a higher DNA content than the uncoated sample. A higher DNA content indicated that the more viable cells were detected on the melt-bonded scaffold in comparison to the untreated nonwoven fabric and the prior art scaffold.

Although this invention has been shown and described with respect to detailed embodiments thereof, it will be understood by those skilled in the art that various changes in form and detail thereof may be made without departing from the spirit and scope of the claimed invention.

We claim:

1. A method of making a hollow organ tissue repair scaffold comprising the steps of:
providing a nonwoven fabric comprising a first biocompatible, bioabsorbable material having a first melting temperature, and a second biocompatible, bioabsorbable material, having a second melting temperature, wherein the first melting temperature is lower than the second melting temperature;
forming the nonwoven fabric into a hollow organ shape substantially in the shape of at least part of a hollow organ;
heating the shaped fabric to a temperature sufficiently effective to at least partially melt the first biocompatible, bioabsorbable material without melting the second biocompatible, bioabsorbable material; and,
allowing the shaped fabric to cool to room temperature, thereby providing a hollow organ tissue engineering repair scaffold.

2. The method of claim 1, wherein the nonwoven fabric comprises staple fibers further comprising a first biocompatible, bioabsorbable material having a first melting temperature, and a second biocompatible, bioabsorbable material, having a second melting temperature, wherein the first melting temperature is lower than the second melting temperature.

3. The method of claim 1, wherein the first biocompatible material comprises poly(p-dioxanone).

4. The method of claim 1, wherein the second biocompatible material comprises an aliphatic polyester homopolymers or copolymers prepared from monomers including, but not limited to, L-lactide, D-lactide, meso-lactide, lactic acid, glycolide, glycolic acid, epsilon-caprolactone, p-dioxanone (1,4-dioxan-2-one), trimethylene carbonate and combinations thereof

5. The method of claim 4, wherein the second biocompatible material comprises an aliphatic polyester homopolymers or copolymers prepared from monomers including, but not limited to, L-lactide, D-lactide, meso-lactide, lactic acid, glycolide, glycolic acid, and combinations thereof.

6. The method of claim 1, wherein the second biocompatible material comprises a polymer selected from the group consisting of poly(glycolide) and poly(glycolide-co-lactide).

7. The method of claim 1, additionally comprising the steps of forming the nonwoven fabric into a hollow organ shape by:
cutting the nonwoven fabric into a suitable shape; and,
approximating edges of the of the shaped nonwoven fabric.

8. The method of claim 1, wherein the shape comprises a shape selected from the group consisting of square, rectangular, triangular, petal, and the like.

9. The method of claim 1, wherein the temperature is about 105° C. to about 150° C.

10. The method of claim 1 wherein nonwoven fabric comprises about about 20% to about 50% by weight of the first biocompatible, bioabsorbable material.

11. The method of claim 1 wherein the hollow organ tissue engineering scaffold has a shape selected from the group consisting of_spherical, prism, cylindrical, and the like.

12. The method of claim 11, wherein the shape is useful to repair an organ selected from the group consisting of_ bladder, urethra, jejunum, esophagus, trachea, colon, blood vessels, stomach, and nerve guides.

13. A hollow organ tissue repair scaffold, made by the method comprising the steps of:
providing a nonwoven fabric comprising a first biocompatible, bioabsorbable material having a first melting temperature, and a second biocompatible, bioabsorbable material, having a second melting temperature, wherein the first melting temperature is lower than the second melting temperature;
forming the nonwoven fabric into a hollow organ shape substantially in the shape of at least part of a hollow organ;
heating the shaped fabric to a temperature sufficiently effective to at least partially melt the first biocompatible, bioabsorbable material without melting the second biocompatible, bioabsorbable material; and,
allowing the shaped fabric to cool to room temperature; thereby providing a hollow organ tissue engineering repair scaffold.

14. The scaffold of claim 13, wherein the nonwoven fabric comprises staple fibers further comprising a first biocompatible, bioabsorbable material having a first melting temperature, and a second biocompatible, bioabsorbable material, having a second melting temperature, wherein the first melting temperature is lower than the second melting temperature.

15. The scaffold of claim 13, wherein the first biocompatible material comprises poly(p-dioxanone).

16. The scaffold of claim 13, wherein the second biocompatible material comprises an aliphatic polyester homopolymers or copolymers prepared from monomers including, but not limited to, L-lactide, D-lactide, meso-lactide, lactic acid, glycolide, glycolic acid, epsilon-caprolactone, p-dioxanone (1,4-dioxan-2-one), trimethylene carbonate and combinations thereof

17. The scaffold of claim 16, wherein the second biocompatible material comprises an aliphatic polyester homopolymers or copolymers prepared from monomers including, but not limited to, L-lactide, D-lactide, meso-lactide, lactic acid, glycolide, glycolic acid, and combinations thereof.

18. The scaffold of claim 13, wherein the second biocompatible material comprises a polymer selected from the group consisting of poly(glycolide) and poly(glycolide-co-lactide).

19. The scaffold of claim 13, additionally comprising the steps of forming the nonwoven fabric into a hollow organ shape by:
cutting the nonwoven fabric into a suitable shape; and,
approximating edges of the shaped nonwoven fabric.

20. The scaffold of claim 13, wherein the shape comprises a shape selected from the group consisting of square, rectangular, triangular, petal, and the like.

21. The scaffold of claim 13, wherein the temperature is about 105° C. to about 150° C.

22. The scaffold of claim 13, wherein nonwoven fabric comprises about 20% to about 50% by weight of the first biocompatible, bioabsorbable material.

23. The scaffold of claim 13, wherein the hollow organ tissue engineering scaffold has a shape selected from the group consisting of spherical, prism, cylindrical, and the like.

24. The scaffold of claim 23, wherein the shape is useful to repair an organ selected from the group consisting of_ bladder, urethra, jejunum, esophagus, trachea, colon, blood vessels, stomach, and nerve guides.
